(19) United States
(12) Patent Application Publication
(10) Pub. No.: US 2020/0323739 A1
(43) Pub. Date: Oct. 15, 2020

(54) SYSTEMS AND METHODS FOR BREAST MILK COLLECTION, STORING, AND FEEDING

(71) Applicant: ReMana, Inc., Boston, MA (US)

(72) Inventors: Lindsay E. MCCURREN, Boston, MA (US); Nathan D. MCCURREN, Boston, MA (US)

(73) Assignee: ReMana, Inc., Boston, MA (US)

(21) Appl. No.: 16/843,696

(22) Filed: Apr. 8, 2020

Related U.S. Application Data
(60) Provisional application No. 62/831,416, filed on Apr. 9, 2019.

(51) Int. Cl. A61J 9/00; A61J 9/08; A61J 11/04; A61M 1/06; A61J 11/00

(52) U.S. Cl. CPC A61J 9/00; A61J 9/085; A61J 11/0085; A61M 1/062; A61J 11/04

(57) ABSTRACT

Embodiments of the systems and methods described herein include a system of improved silicone bottle containers, adapters, and nipples that support the collection, storage, and feeding of breast milk. In some embodiments, a bottle system may include a bottle formed from molded silicone rubber including a bottom end having a recess, and a top end having an opening configured to receive and dispel contents, wherein the top end is spaced apart from the bottom end, and an inner adapter composed of molded standard thermo-plastic configured to engage with the top end of the bottle. The bottle system may also include a flat cap, nipple, nipple cover, nipple adapter, and/or breast pump adapter.

### CROSS REFERENCE TO RELATED APPLICATIONS

This application claims priority to U.S. Provisional Application No. 62/831,416 filed Apr. 9, 2019 entitled “SYSTEMS AND METHODS FOR BREAST MILK COLLECTION, STORING, AND FEEDING,” the contents of which is hereby incorporated by reference.

### TECHNICAL FIELD

The present disclosure is directed towards systems and methods for breast milk collection, storing, and feeding.

### BACKGROUND

Conventional systems for the collection, storing, and feeding of breast milk present many challenges.

Mothers may often use breast pumps to express breast milk that may be used at a later time. For example, in conventional systems, mothers typically breast pump into round plastic bottles and then transfer it to plastic storage bags that are then placed into a freezer for storage and then retrieved and thawed at a later time. The conventional plastic bags are difficult to handle and inconvenient to store in an organized way. Because the bags are not organized, many times they can be lost within a freezer. Frequent movement and rearranging within the freezer often leads to tears in the bags, especially in the corners, which then leak on thawing.

Additionally, in conventional plastic bags, the breast milk may be defrosted by placing the plastic bag in the refrigerator or using warm/hot water. However, this heating may lead to leaching of the chemical materials used to construct the plastic bags into the breast milk, which may pose risks to the baby.

Further, conventional systems used for storing and feeding of breast milk, require the transfer of breast milk from the plastic bags to a bottle that is capable of feeding a baby. This transfer point is an opportunity to spill milk. After transfer, the plastic bag is discarded after just a single use as it cannot be sterilized. Often, conventional bottles are not configured to provide a pleasant experience for the baby, who may have trouble latching onto the nipple, and/or experience problems with gas, depending on how the bottle is configured to let air in.

### SUMMARY

Disclosed herein are systems and methods for breastmilk collection, storing, and feeding. In some embodiments, the improved systems and methods include a silicone based bottle system including bottles, adapters, nipples, nipple covers, and the like. Embodiments of the present disclosure may address one or more of the problems discussed above.

In some embodiments, a bottle system may include a bottle formed from molded silicone rubber including a bottom end having a recess, and a top end having an opening configured to receive and dispel contents, wherein the top end is spaced apart from the bottom end, and an inner adapter composed of standard plastic configured to engage with the top end of the bottle. In some embodiments, the inner adapter may be shaped as an o-ring shaped collar. The bottle system may also include a flat cap configured to engage with the inner adapter and form a closed environment, the flat cap having a protruding surface configured to engage with the recess of the bottle. The bottle system may also include a nipple configured to engage with an upper surface of the bottle opening. In some embodiments, the bottle system may also include a nipple adapter configured to engage with a nipple and secure the nipple to the inner adapter. In some embodiments, the bottle system may also include a nipple cover configured to engage with the nipple adapter and prevent the nipple from expelling liquid contained within the bottle, where the nipple cover includes a protruding surface configured to engage with the recess of the bottle. Further, in some embodiments the bottle system may include a storage element having a first end spaced apart from a second end, where the first end has a greater height than the second end, and the storage element is configured to store a plurality of bottles. In some embodiments, the nipple may be ventless. In alternative embodiments, the nipple may include at least one vent.

In some embodiments, a bottle system may include breast pump adapters configured to connect to a particular breast pump and having a first end designed to attach to a pump valve of the breast pump and a second end configured to attach to the inner adapter.

### BRIEF DESCRIPTION OF DRAWINGS

The accompanying drawings, which are incorporated in and constitute a part of this specification, illustrate several embodiments and, together with the description, serve to explain the disclosed principles. In the drawings:

FIG. 1A illustrates a first side perspective view of a bottle in accordance with some embodiments of the present disclosure.

FIG. 1B illustrates a second side perspective view of a bottle in accordance with some embodiments of the present disclosure.

FIG. 1C illustrates a cross-sectional view of a bottle in accordance with some embodiments of the present disclosure.

FIG. 2A illustrates a first side perspective view of a bottle in accordance with some embodiments of the present disclosure.

FIG. 2B illustrates a second side perspective view of a bottle in accordance with some embodiments of the present disclosure.

FIG. 2C illustrates a cross-sectional view of a bottle in accordance with some embodiments of the present disclosure.

FIG. 3A illustrates a first perspective view of an inner adapter for a bottle in accordance with some embodiments of the present disclosure.

FIG. 3B illustrates a second perspective view of an inner adapter for a bottle in accordance with some embodiments of the present disclosure.

FIG. 3C illustrates a cross-sectional view of an inner adapter for a bottle in accordance with some embodiments of the present disclosure.

FIG. 4A illustrates a first perspective view of an outer cap for a bottle in accordance with some embodiments of the present disclosure.

FIG. 4B illustrates a second perspective view of an outer cap for a bottle in accordance with some embodiments of the present disclosure.

FIG. 4C illustrates a cross-sectional view of an outer cap for a bottle in accordance with some embodiments of the present disclosure.

FIG. 5A illustrates a first perspective view of an assembly including a bottle and an outer cap in accordance with some embodiments of the present disclosure.

FIG. 5B illustrates a second perspective view of an assembly including a bottle and an outer cap in accordance with some embodiments of the present disclosure.

FIG. 5C illustrates a cross-sectional view of an assembly including a bottle and an outer cap in accordance with some embodiments of the present disclosure.

FIG. 6A illustrates a first perspective view of a nipple for a bottle in accordance with some embodiments of the present disclosure.

FIG. 6B illustrates a cross-sectional view of a nipple for a bottle in accordance with some embodiments of the present disclosure.

FIG. 6C illustrates a second cross-sectional view of a nipple for a bottle in accordance with some embodiments of the present disclosure.

FIG. 7A illustrates a first perspective view of a nipple adapter for a bottle in accordance with some embodiments of the present disclosure.

FIG. 7B illustrates a second perspective view of a nipple adapter for a bottle in accordance with some embodiments of the present disclosure.

FIG. 7C illustrates a cross-sectional view of a nipple adapter for a bottle in accordance with some embodiments of the present disclosure.

FIG. 8A illustrates a first perspective view of an assembly including a bottle, nipple, and a nipple adapter in accordance with some embodiments of the present disclosure.

FIG. 8B illustrates a second perspective view of an assembly including a bottle, nipple, and a nipple adapter in accordance with some embodiments of the present disclosure.

FIG. 8C illustrates a third perspective view of an assembly including a bottle, nipple, and a nipple adapter in accordance with some embodiments of the present disclosure.

FIG. 8D illustrates a fourth perspective view of an assembly including a bottle, nipple, and a nipple adapter in accordance with some embodiments of the present disclosure.

FIG. 8E illustrates a fifth perspective view of an assembly including a bottle, nipple, and a nipple adapter in accordance with some embodiments of the present disclosure.

FIG. 8F illustrates a sixth perspective view of an assembly including a bottle, nipple, and a nipple adapter in accordance with some embodiments of the present disclosure.

FIG. 9A illustrates a first perspective view of a nipple cover for a bottle in accordance with some embodiments of the present disclosure.

FIG. 9B illustrates a second perspective view of a nipple cover for a bottle in accordance with some embodiments of the present disclosure.

FIG. 9C illustrates a cross-sectional view of a nipple cover for a bottle in accordance with some embodiments of the present disclosure.

FIG. 10A illustrates a first perspective view of an assembly including a bottle, nipple, nipple cover and a nipple adapter in accordance with some embodiments of the present disclosure.

FIG. 10B illustrates a second perspective view of an assembly including a bottle, nipple, nipple cover and a nipple adapter in accordance with some embodiments of the present disclosure.

FIG. 10C illustrates a third perspective view of an assembly including a bottle, nipple, nipple cover and a nipple adapter in accordance with some embodiments of the present disclosure.

FIG. 10D illustrates a fourth perspective view of an assembly including a bottle, nipple, nipple cover and a nipple adapter in accordance with some embodiments of the present disclosure.

FIG. 11 illustrates a perspective view of an assembly including a bottle, nipple, nipple cover and a nipple adapter in accordance with some embodiments of the present disclosure.

FIG. 12A illustrates a first perspective view of a breast pump adapter for a bottle in accordance with some embodiments of the present disclosure.

FIG. 12B illustrates a second side perspective view of a breast pump adapter for a bottle in accordance with some embodiments of the present disclosure.

FIG. 12C illustrates a third perspective view of a breast pump adapter for a bottle in accordance with some embodiments of the present disclosure.

FIG. 12D illustrates a cross-sectional perspective view of a breast pump adapter for a bottle in accordance with some embodiments of the present disclosure.

FIG. 13A illustrates a first perspective view of a an assembly including a bottle and breast pump adapter in accordance with some embodiments of the present disclosure.

FIG. 13B illustrates a second perspective view of a an assembly including a bottle and breast pump adapter in accordance with some embodiments of the present disclosure.

FIG. 13C illustrates a third perspective view of a an assembly including a bottle and breast pump adapter in accordance with some embodiments of the present disclosure.

FIG. 13D illustrates a cross-sectional view of a an assembly including a bottle and breast pump adapter in accordance with some embodiments of the present disclosure.

FIG. 14A illustrates a first view of an assembly including various bottles, and a storage element in accordance with some embodiments of the present disclosure.

FIG. 14B illustrates a second view of an assembly including various bottles, and a storage element in accordance with some embodiments of the present disclosure.

FIG. 14C illustrates a third view of an assembly including various bottles, and a storage element in accordance with some embodiments of the present disclosure.

FIG. 14D illustrates a fourth view of an assembly including various bottles, and a storage element in accordance with some embodiments of the present disclosure.

FIG. 14E illustrates a fifth view of an assembly including various bottles, and a storage element in accordance with some embodiments of the present disclosure.

FIG. 15A illustrates a first perspective view of an assembly including stacked bottles in accordance with some embodiments of the present disclosure.

FIG. 15B illustrates a second perspective view of an assembly including stacked bottles in accordance with some embodiments of the present disclosure.

FIG. 16 illustrates a first perspective view of an assembly including stacked bottles in accordance with some embodiments of the present disclosure.

### DETAILED DESCRIPTION

Embodiments of the systems and methods described herein include a system of improved silicone bottle containers, adapters, and nipples that support the collection, storage, and feeding of breast milk. Embodiments of the system may enable parents to efficiently handle and organize breastmilk from the time it leaves the mother's breast to the time it enters a baby's mouth. The system collects breastmilk as it is pumped, stores the breastmilk and then may also be used to feed a baby.

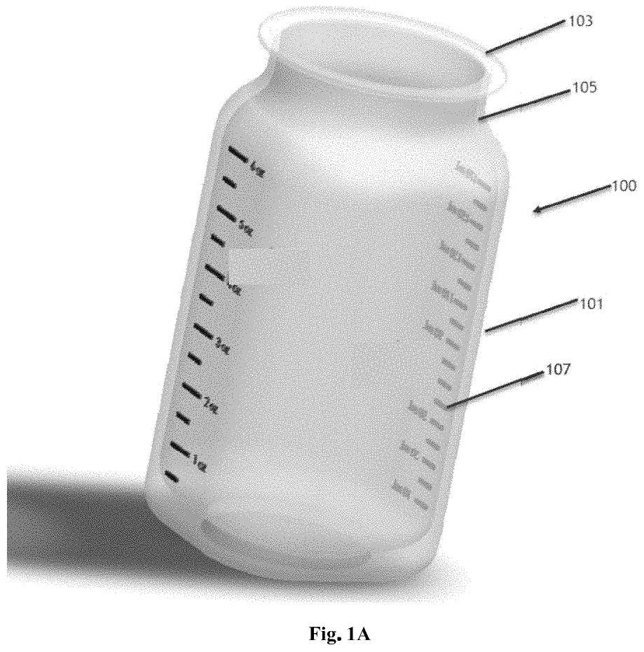

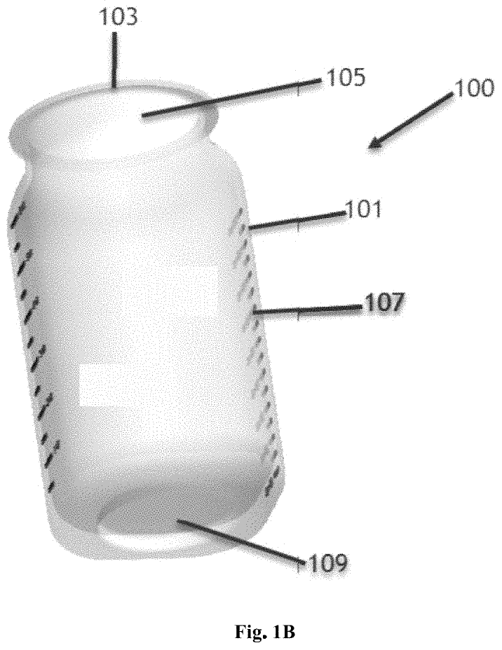

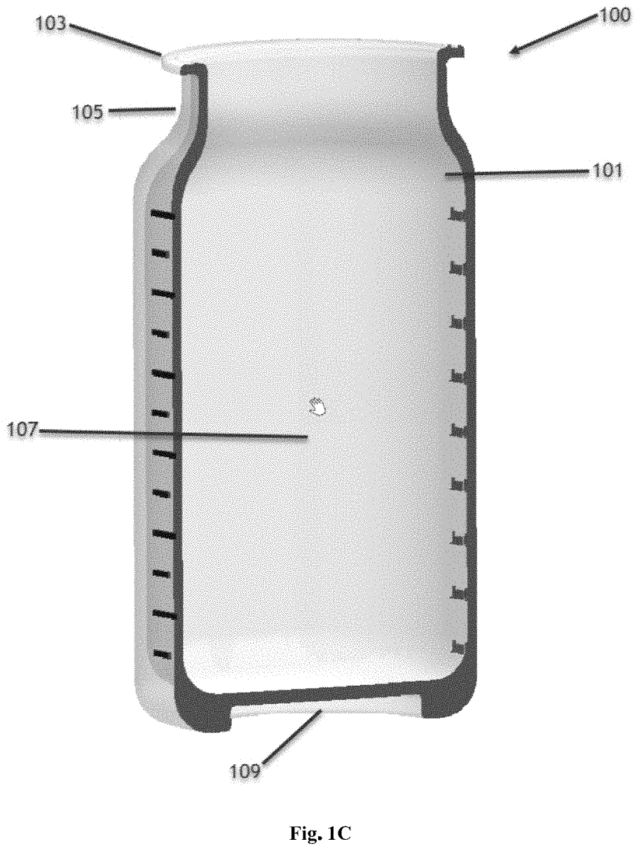

FIGS. 1A-1C illustrate various views of a bottle in accordance with at least one embodiment of the present disclosure. In particular, FIG. 1A provides a top perspective view, FIG. 1B provides a bottom perspective view, and FIG. 1C provides a cross-sectional view of a bottle 100. A bottle 100 may include an exterior 101, is configured to be filled with contents (e.g., milk, breast milk) 107. A first end of the bottle may have an opening surrounded by a top lip 103 that is spaced apart 105 from the main portion of the bottle. The opening may be sized to be smaller than the width of the bottle. As illustrated in FIG. 1B, a lower surface of the bottle may include a recessed region 109, sized and configured to receive a top surface/cap of another bottle. Accordingly, the bottles may be stacked. In some embodiments, the exterior 101 of the bottle may be marked with one or more scales (e.g., milliliters, ounces).

A bottle such as bottle 100 may be of any suitable size. In some embodiments, a bottle may be configured to have a capacity of approximately 4 ounces. In some embodiments, a bottle may be configured to have a capacity of approximately 6 ounces. In other embodiments, a bottle may be configured to have a capacity of approximately 8 ounces Other capacities are envisioned. A system built in accordance with the disclosed embodiments, may include a number of bottles having the same or varied capacities.

In some embodiments, the bottle may be formed of injection molded liquid silicone rubber. In some embodiments, the bottle may be formed of medical and food grade liquid silicone rubber. Advantageously, in comparison with conventional bottles, the disclosed bottles including injected molded liquid silicone rubber may be safer for contact with food, and breast milk. As silicone is an inert chemical (as opposed to a synthetic plastic), it is unlikely that the silicone will leach chemicals into the contents of the bottle (e.g., breast milk), even when heated. Additionally, silicone includes anti-microbial properties, which may assist in preventing the growth of bacteria within the bottle. Further, because silicone can withstand a wide temperature range, bottles built in accordance with the present disclosure may be stored in freezing temperatures (i.e., in a freezer), be heated/defrosted (i.e., in warm water, in a refrigerator), and sterilized using a dishwasher. Accordingly, bottles formed from molded silicone rubber may present many advantages over conventional bottles that may include plastics, plastics having BPA, and the like.

In addition to the benefits described above, in some embodiments, the bottle may be shaped in order to provide an ergonomic and aesthetic benefit to a baby. In comparison to plastic, silicone is softer and easier for a baby to hold. Additionally, because silicone is soft, and non-rigid, the silicone more closely mimics nursing from mom and provides a baby with a more natural feeding experience.

In some embodiments, a bottle may have any suitable elongated prism shape that is substantially cylindrical. For example, the bottle containers may have a modular, super-elliptical, squared circle, or squircle shape. In some embodiments, the shape of the bottle (across a horizontal cross section) may form a gentle square body. The shape of the bottle (e.g. gentle square body) may provide more efficient storage of the bottle in comparison with conventional bottles. Because of the modular shape, the sides of bottles will align flush to one another when stored. In other words, the bottles can be stored adjacent to each other, without requiring a separating space. In conventional systems, having round bottles, there is dead space created in between the circular containers that does not allow for close configurations and leads to inefficient storage.

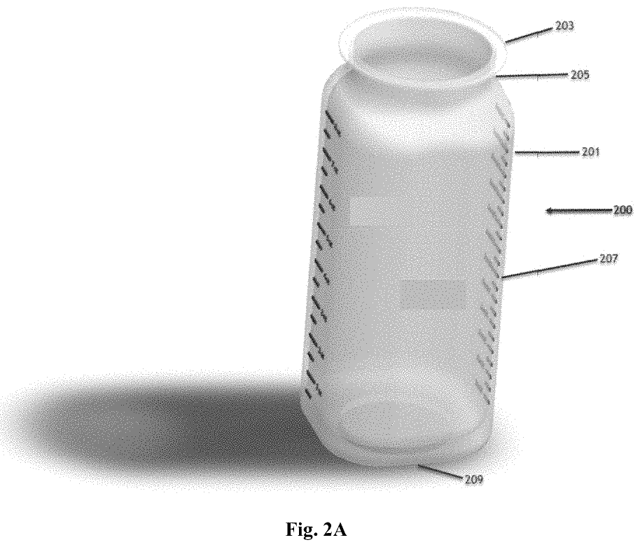

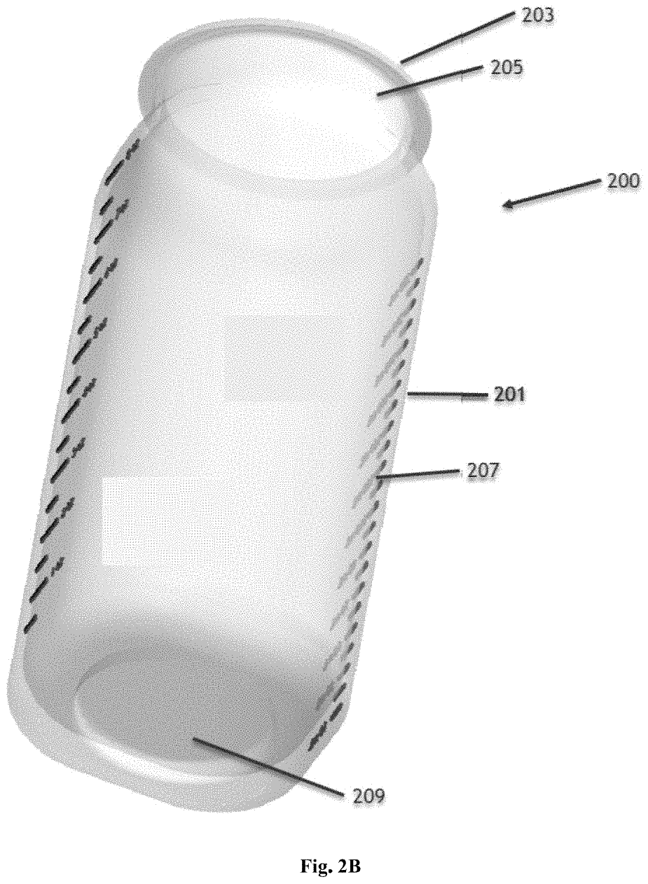

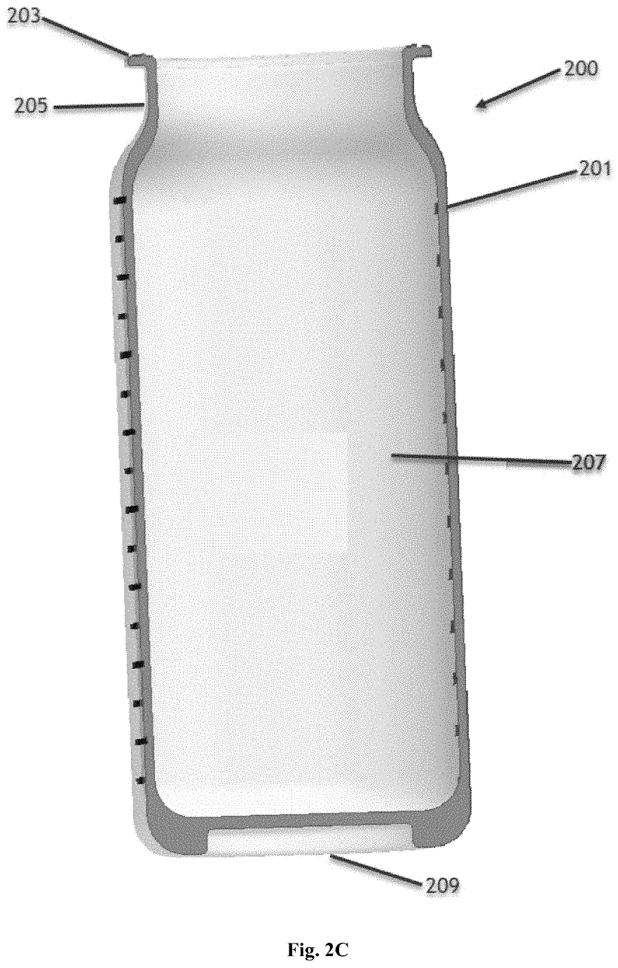

FIGS. 2A-2C illustrate various views of a bottle in accordance with at least one embodiment of the present disclosure. The bottle 200 depicted in FIGS. 2A-2C is shown to be a larger bottle (having more capacity) than the bottle depicted in FIGS. 1A-1C. In particular, FIG. 2A provides a top perspective view, FIG. 2B provides a bottom perspective view, and FIG. 2C provides a cross-sectional view of a bottle 200. A bottle 200 may include an exterior 201, is configured to be filled with contents (e.g., milk, breast milk) 207. A first end of the bottle may have an opening surrounded by a top lip 203 that is spaced apart 205 from the main portion of the bottle. The opening may be sized to be smaller than the width of the bottle. As illustrated in FIG. 2B, a lower surface of the bottle may include a recessed region 209, sized and configured to receive a top surface/cap of another bottle. Further, in some embodiments, a bottle 100 of FIG. 1 may be compatible to be stacked with a bottle 200 of FIG. 2. Accordingly, the bottles may be stacked. In some embodiments, the exterior 201 of the bottle may be marked with one or more scales (e.g., milliliters, ounces).

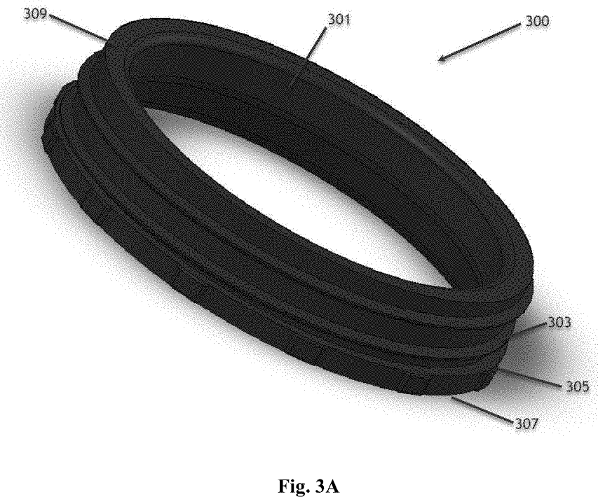

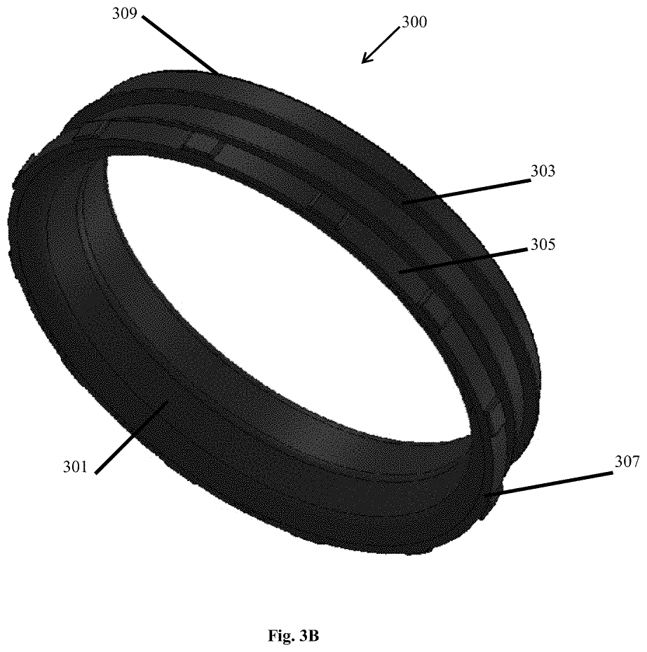

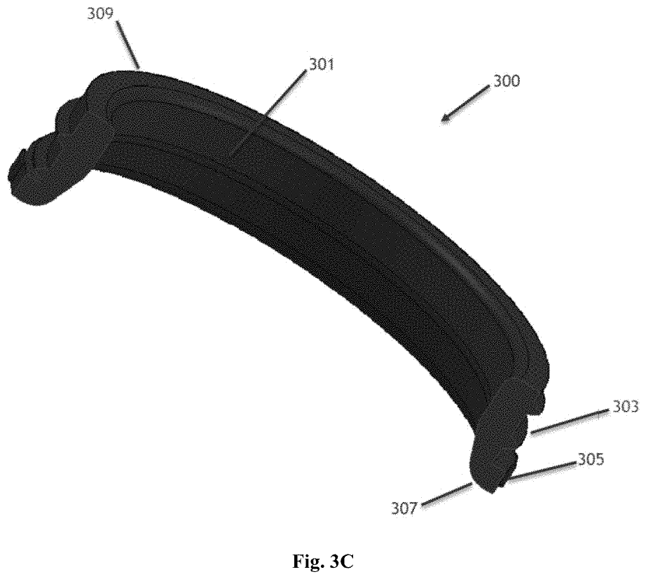

FIGS. 3A-3C illustrate various views of an inner adapter (also referred to as an inner cap, o-ring shaped collar) for a bottle in accordance with at least one embodiment of the present disclosure. In particular, FIG. 3A provides a top perspective view, FIG. 3B provides a bottom perspective view, and FIG. 3C provides a cross sectional view of the inner adapter 300. In some embodiments, the inner adapter 300 may be an o-ring shaped collar. As illustrated, an inner adapter 300 may have a substantially circular shape, having a hollow interior. The inner adapter 300 may include an interior surface 301, an exterior surface 303 including attachment means, a ridge/lip 305, a lower face 307, and a top surface 309. In some embodiments, the attachment means of the exterior surface 303 includes a male screw pattern.

In some embodiments, the interior surface 301 of the inner adapter 300 may be configured to fit around the opening of the first end of the bottle (e.g. 100, 200). For example, the top lip of the bottle 103, 203 may be positioned above the inner adapter such that the interior surface 301 of the inner adapter may be flush with the space 105, 205.

The inner adapter may be configured from silicone having the same or different properties of the silicone of the bottle 100, 200. In some embodiments, the inner adapter may be configured from standard injected plastic. The inner adapter may be configured to allow attachment to a breast pump adapter, a nipple cap, a flat outer cap, and the like (described below). The top lip 103, 203 of the bottles may include a ridge or lip formed of silicone that is configured to create a liquid tight seal. As opposed to conventional systems, where an outer cap may interface with a flat surface, and may drag when twisting on a cap, the ridge or lip 103 of the bottle container 100, 200 provides many advantages. For example, the ridge or lip 103, 203 provides a smaller contact point for any attaching elements such as adapters, nipples, outer caps and the like that interfaces with the inner adapter. Further, because the ridge or lip 103, 203 may be formed of silicone, the ridge or lip 103, 203 may be compressed (or squished) when an attaching element is tightened about the ridge or lip 103, 203. Compression of the ridge or lip 103, 203 against the top surface 309 of the inner adapter may provide an extra tight seal and aid in preventing the inadvertent expelling of contents from the bottle 100, 200. The tight seal may be configured to prevent the exchange of air and/or liquid from the bottle and the exterior of the bottle. In some embodiments, the seal is specifically formed by a small protrusion in ridge or lip 103, 203 that prevents that surface from being too flat. The protrusion may be minor, and configured to protrude on the order of 0-3 millimeters. In some embodiments, the compression of silicone into itself when the threads of the inner adapter 303 are matched with any interfacing piece, and creates the liquid tight seal referenced above.

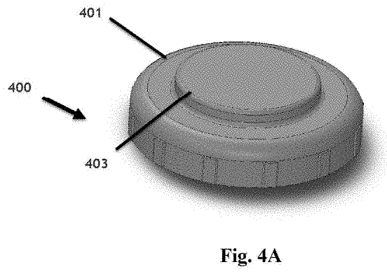

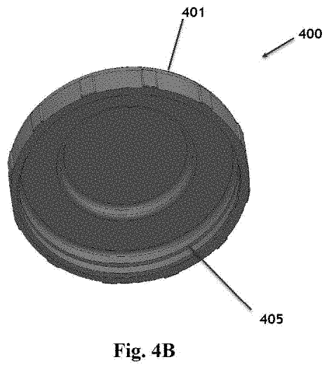

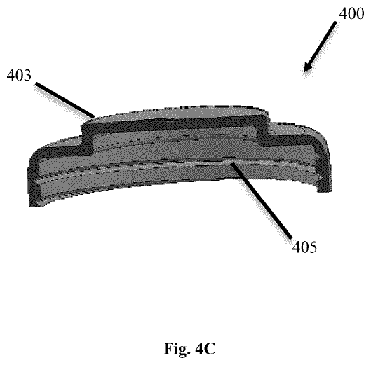

FIGS. 4A-4C illustrate various views of an outer cap for a bottle in accordance with at least one embodiment of the present disclosure. In particular, FIG. 4A illustrates a top perspective view, FIG. 4B illustrates a bottom perspective view, and FIG. 4C illustrates a cross-sectional view. The outer cap (also referred to as a flat cap) 400, may include a raised portion 403, an exterior surface 401, and an interior attachment means 405.

In some embodiments, the outer cap 400 may be formed of thermo-plastic. The outer cap 400 may engage with the inner adapter 300 by engaging the interior attachment means 405 of the outer cap 400 with the exterior surface 303 of the inner adapter 300. The outer cap 400 may be configured to keep the contents of the bottle 100, 200 from being exposed. The outer cap 400 may also function to create a liquid tight container, thus allowing the container or bottle to be capable of being transported.

In some embodiments, the interior attachment means 405 includes a complementary component to the exterior surface 303 of the inner adapter 300. For example, if the inner adapter 300 is configured with an exterior surface 303 having a male thread, the interior attachment means 405 of the outer cap 400 may include a female thread capable of engaging with the male thread.

In some embodiments, the outer cap 400 includes a raised portion 403 that may be configured to have a shape complementary to the recess 109, 209, positioned on the bottom of the bottle 100, 200. Accordingly, the outer cap may include means for stacking one bottle on top of another.

In some embodiments, the exterior surface 401 of the outer cap 400 may include rivets or extruded ridges around the perimeter of the outer cap 400 that may be configured to allow the human hand something to grip onto when engaging or disengaging the outer cap 400 with the inner adapter 300. The rivets or extruded ridges may allow a user to achieve an extra tight closure on the outer cap 400, and compress the extra silicone on the lip 305 to form a liquid tight seal.

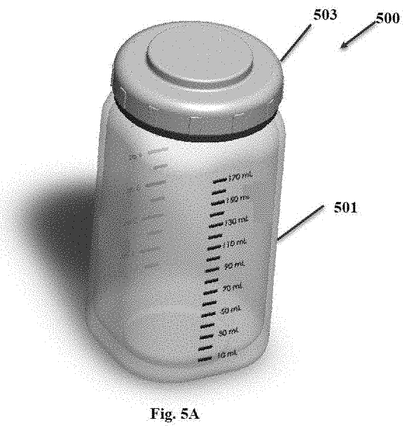

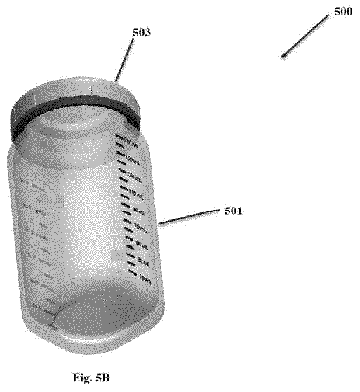

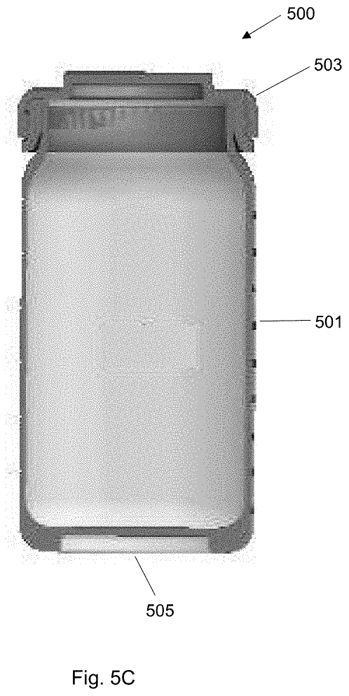

FIGS. 5A-5C illustrate various views of an assembly 500 including a bottle 501 and an outer cap 503 in accordance with at least one embodiment of the present disclosure. In particular, FIGS. 5A and 5B provide perspective views of the assembly, and FIG. 5C provides a cross-sectional view. As illustrated in the cross-sectional view of FIG. 5C, the outer cap may engage with the inner adapter to form a closed container. Further, the bottom surface of the bottle 501 may include a recess 505.

The assemblies illustrated in FIG. 5A-5C may be capable of stacking on top of each other, and may be configured to stay in place by way of the respective raised portions of the outer cap. As described above, the raised portions may engage with recessed portions at the bottom of the bottles.

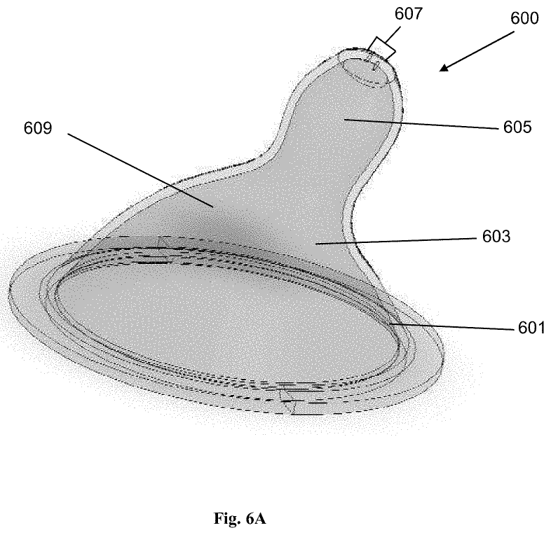

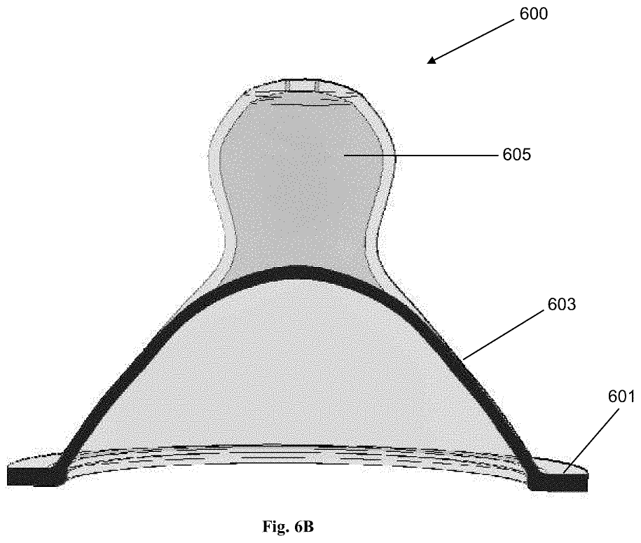

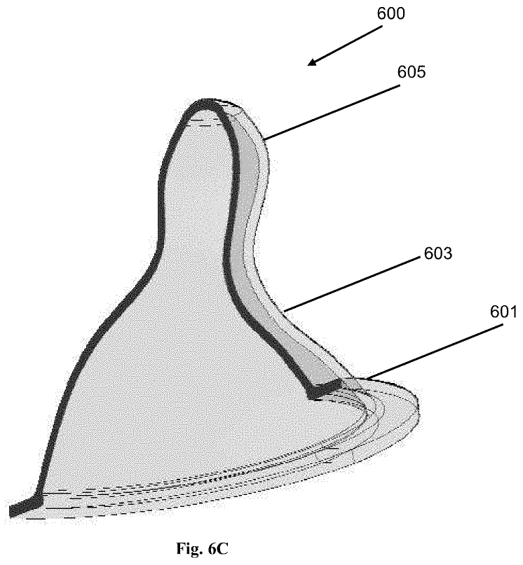

FIGS. 6A-6C illustrate various views of a nipple 600 for a bottle (such as bottle 100, 200), in accordance with at least one embodiment of the present disclosure. In particular, FIG. 6A provides a perspective view of the nipple 600, and FIGS. 6B and 6C provide cross-sectional views of the nipple 600. Nipple 600 may include a lower nipple portion 603, and a upper nipple portion 605.

In some embodiments, the nipple 600 may be formed of silicone.

In some embodiments, the nipple 600 may be shaped to mimic the natural breast shape and nipple profile of a mother. In particular, the nipple 600 may include an asymmetric curve, with the teat portion 607 positioned on an upper nipple portion 605 that is positioned off centered from the lower nipple portion 603. Advantageously, this may allow a baby to drink from the bottle without having to be tipped so far back. Accordingly, the nipple may provide the baby with a more natural way to drink and swallow, than having to lie down completely.

As illustrated in FIGS. 6A-6C, in some embodiments, the teat portion 607 may be designed to be wider (across the palate of the baby) than it is tall (distance from palate to tongue) in order to imitate a more natural shape of mother's nipple when baby is nursing. The flatness of the nipple 600 may also allow baby to compress the nipple 600 more easily and provide the baby with easier to control the flow of breast milk.

As illustrated in FIGS. 6A-6C, the nipple 600 may not contain a vent that allows air to enter the bottle while the baby is drinking. By not including a vent in the nipple, this creates a ventless bottle assembly where no air will enter the bottle while the baby is eating, creating a vacuum. In embodiments made from soft silicone, the vacuum may cause the bottle to collapse slightly while baby eats. Accordingly, while eating, the baby will take in only the small amounts of air that would be in bottle to begin with. This may give the baby a more pleasant eating experience and lead to less gas for the baby.

Alternatively, the nipple 600 may include a vent positioned along the lower nipple portion 603 (not shown). In some embodiments, the vent may be positioned opposite of the teat portion 607. The vent may be configured to allow air to enter during the feeding and assist in preventing the formation of a vacuum inside the bottle. The formation of the vacuum could make it difficult for the baby to extrude milk from the bottle. The positioning of the vent opposite the teat portion 707 may be configured such that air is able to enter the bottle away from where the baby is swallowing, thereby helping reduce the amount of air swallowed immediately by the baby. In some embodiments, the vent may be manufactured using a small laser cut. In other embodiments, the vent may be injection molded. The vent may be further configured to allow air in without letting liquid out.

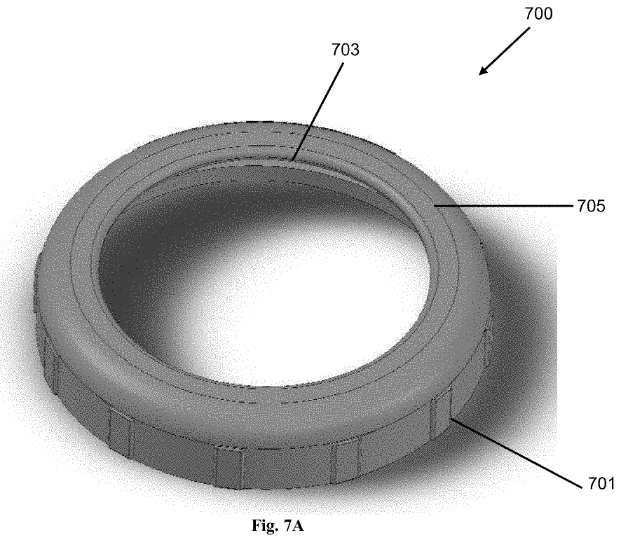

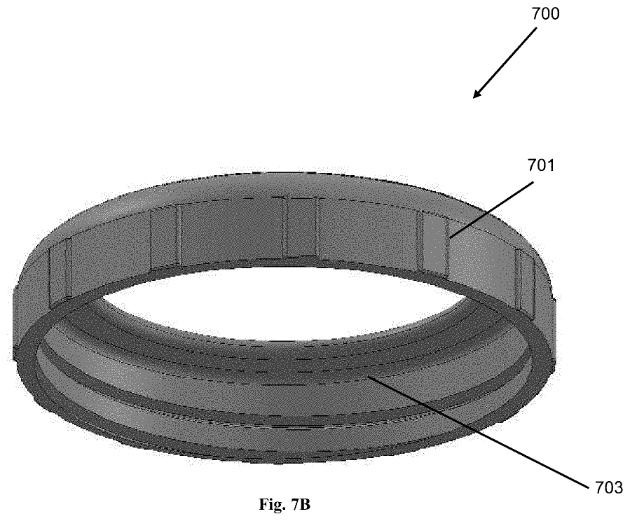

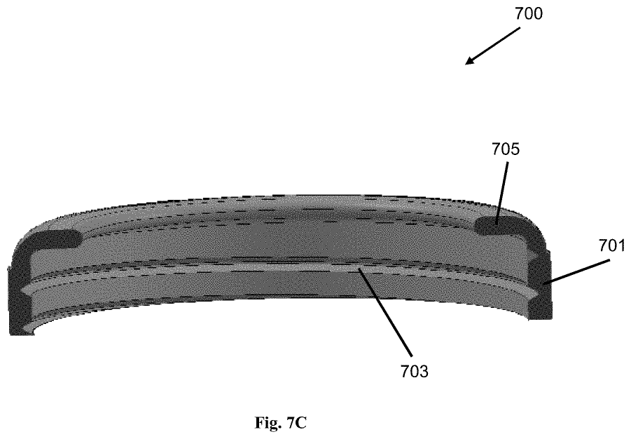

Further, the lower nipple portion 603 may include a lip 601 positioned around the lower circumference of the lower nipple portion 603. When the nipple 600 is engaged with the bottle, the nipple 600 may be positioned such that the lower surface of the lip 601 engages with a top surface of the top lip 103, 203 of the bottle 100, 200. The nipple 600 may be securely attached to the bottle 100, 200 by way of the nipple adapter discussed in connection with FIGS. 7A-7C. In some embodiments, the nipple 600 may be flexible, having a shore durometer of 40A. In some embodiments, the nipple will be 2-3 mm thick. Additionally, the holes in the teat portion of the nipple may be laser cut for precision at diameters less than ⅓ of a millimeter.

FIGS. 7A-7C illustrate various views of a nipple adapter 700 (alternatively referred to as a nipple collar) for a bottle in accordance with at least one embodiment of the present disclosure. In particular, FIGS. 7A and 7B provide perspective views of a nipple adapter 700, and FIG. 7C provides a cross-sectional view of a nipple adapter 00. The nipple adapter 700 may include an outer surface 701, an inner threaded surface 703, and an upper surface 705. The nipple adapter 700 may include a sufficiently hollow center, that is configured to fit a nipple such as nipple 600. A nipple adapter 700 may be configured to securely engage a nipple such as nipple 600 with a bottle such as bottle 100, 200. In particular, the nipple adapter 700 may be configured such at that the upper surface 705 of the nipple adapter fits along the lower surface of the lip 701. Further, the inner threaded surface 703 of the nipple adapter 700 may be configured to be a complementary component to the exterior surface 303 of the inner adapter 300. For example, if the inner adapter 300 is configured with an exterior surface 303 having a male thread, the inner threaded surface 703 of the nipple adapter 700 may include a female thread capable of engaging with the male thread.

Additionally, the outer surface 701 may include various ridges configured to allow a user to grip the nipple adapter 700 to aid in engaging and disengaging the nipple adapter 700 (and nipple 600) from the bottle.

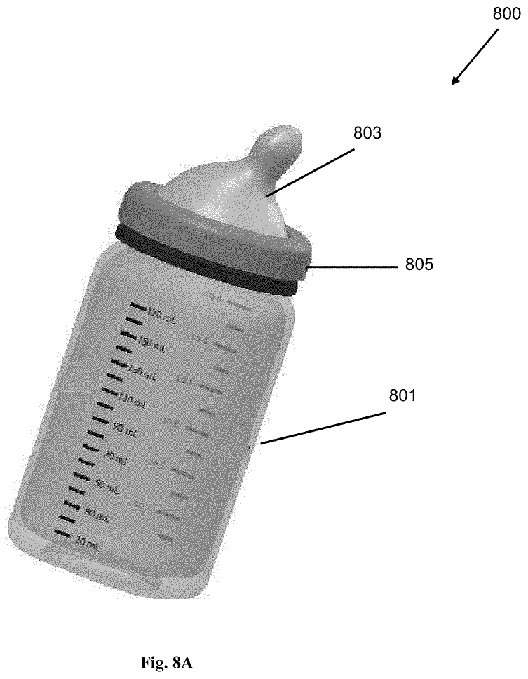

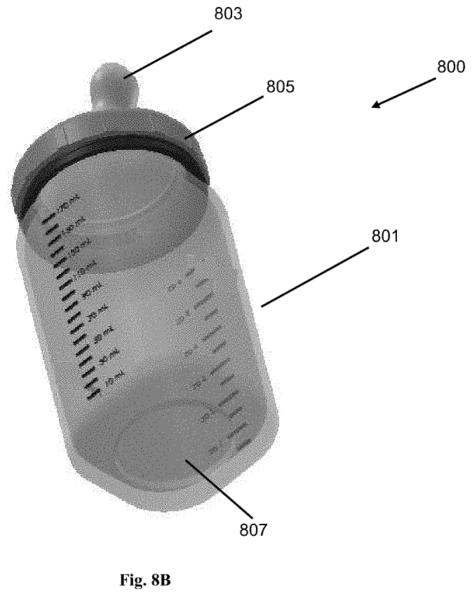

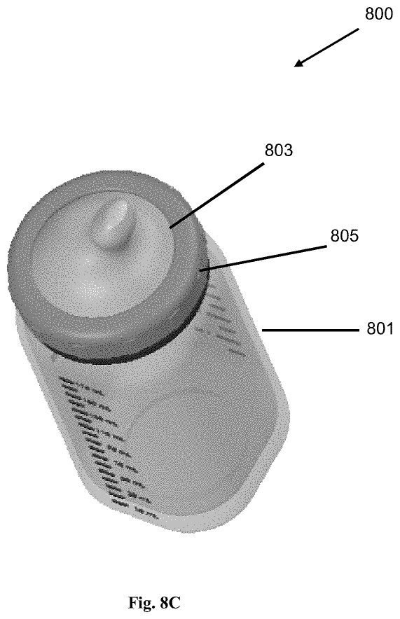

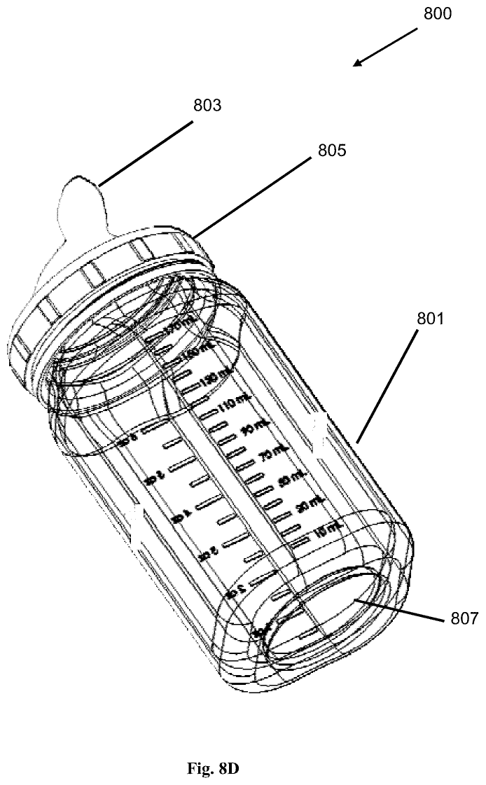

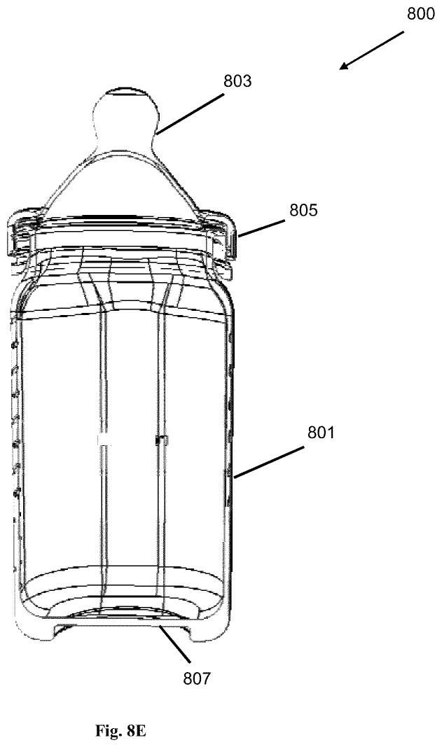

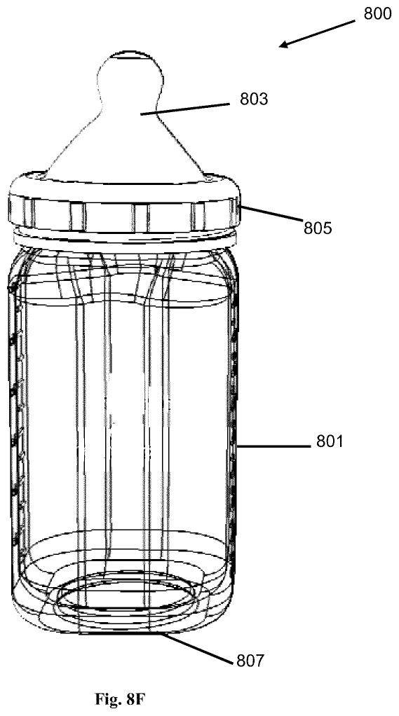

FIGS. 8A-8F illustrate various views of an assembly 800 including a bottle 801, nipple 803, and a nipple adapter 805 in accordance with at least one embodiment of the present disclosure. In particular, FIGS. 8A-8D illustrate various perspective views of the assembly 800, FIG. 8E provides a cross-sectional view of the assembly 800, and FIG. 8F provides a side view of the assembly 800. In particular, the cross-sectional view of FIG. 8E illustrates the engagement of the nipple 803 with the bottle 801, and the nipple adapter 805 with the nipple 803 and the bottle 801. Additionally, a recess 807 in the lower surface of the bottle 801 is visible. In some embodiments the recess 807 may be used for stacking bottles.

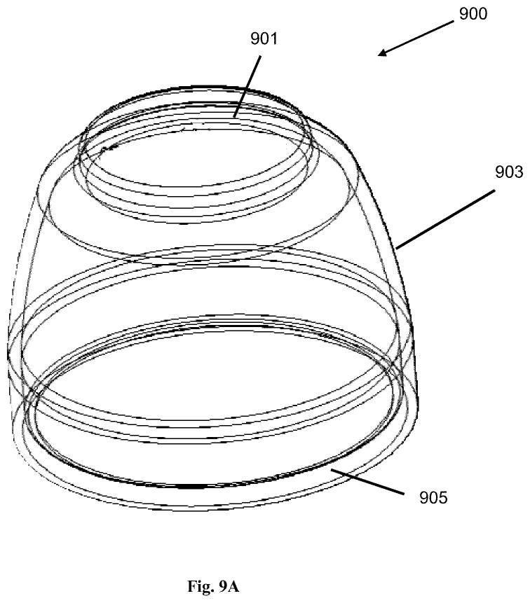

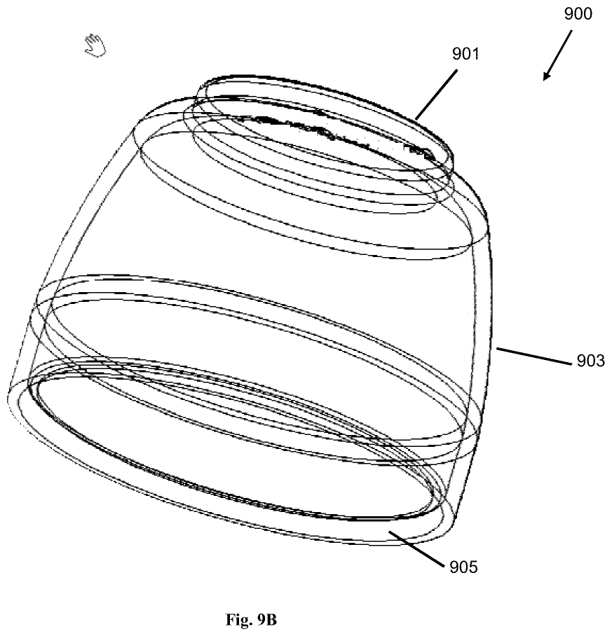

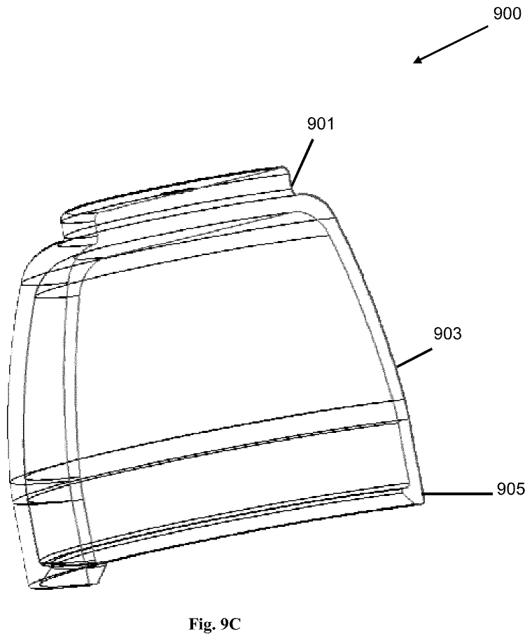

FIGS. 9A-9C illustrate various views of a nipple cover 900 for a bottle in accordance with at least one embodiment of the present disclosure. In particular, FIGS. 9A-9B provide perspective views of the nipple cover 900, and FIG. 9C illustrates a cross-sectional view of the nipple cover 900. In some embodiments, the nipple cover 900 may be formed of clear plastic. The nipple cover 900 may include a protruding nesting feature 901 that is configured to engage with a recess positioned along the bottom surface of the bottle, such as 109, 209, 505, 605, and 807. The nipple cover 900 may also include a surface 903, and a lower rim 905. The nipple cover 900 may also attach to the nipple adapter 805 to form a secure fit. In some embodiments, the nipple cover 900 may attach to the nipple adapter 805 using a snap fit.

In some embodiments, the nipple cover 900 may be formed from a hard, translucent thermo-plastic. The nipple cover 900 may be configured to fit over a bottle when the nipple and nipple collar are attached. In some embodiments, the nipple cover 900 may form a tight fit with the bottle such that it plugs the holes in the teat portion of the nipple where the milk comes out. This allows the bottle to be transported without leaking.

In some embodiments, the nipple cover 900 has a flat upper surface with a protruding nesting feature 901 that is a substantially circular extrusion that allows for nesting of a bottle container on top of it. Accordingly, the bottle to be stacked while in “feeding mode.” In other words, because the clear nipple cover 900 also has the protruding nesting feature 901, the bottles can efficiently be stored in “feeding mode” with a nipple on.

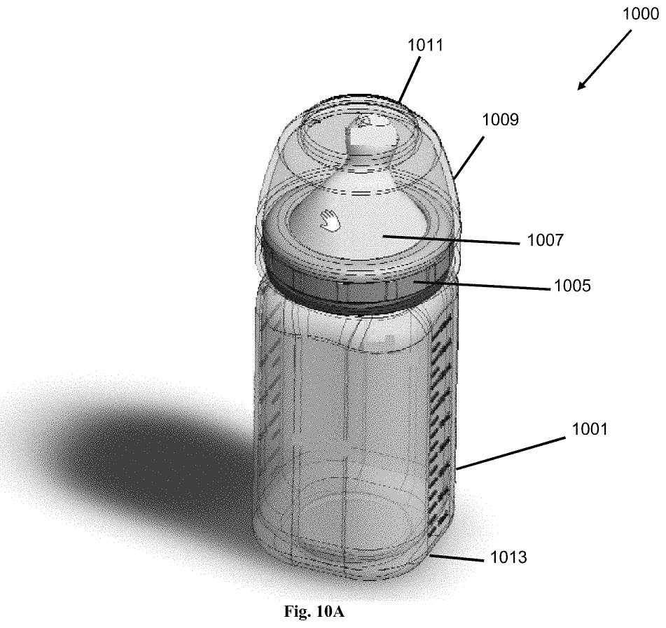

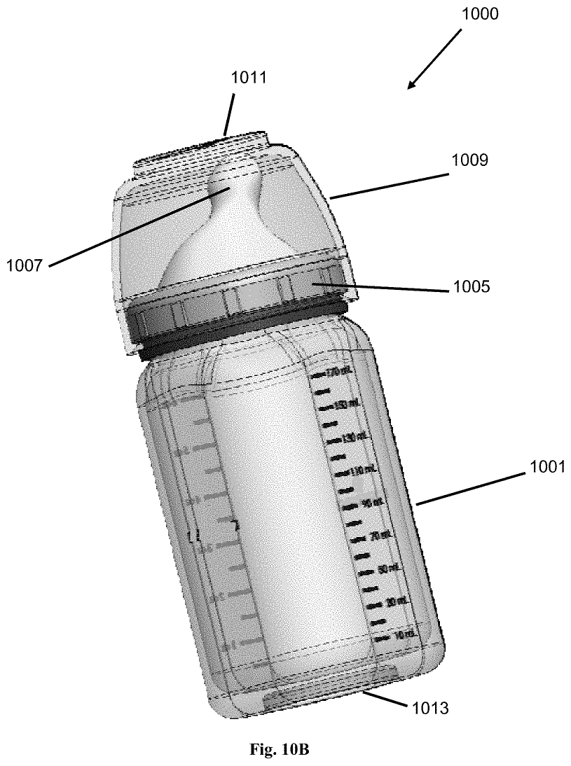

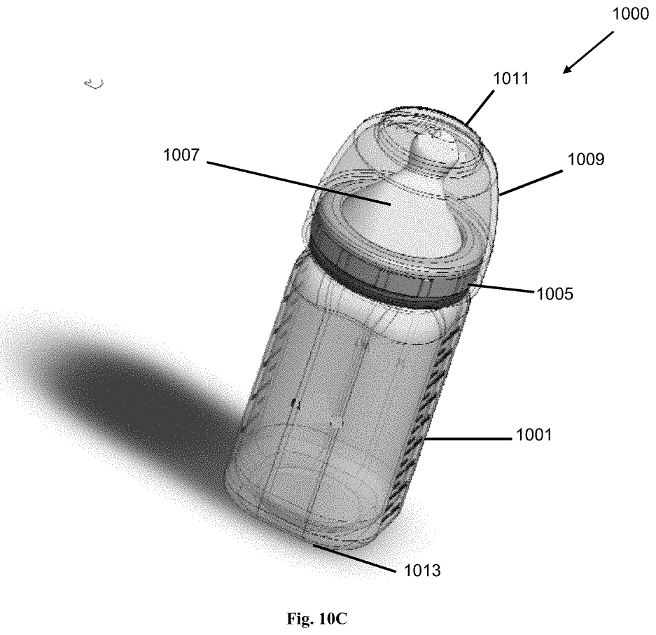

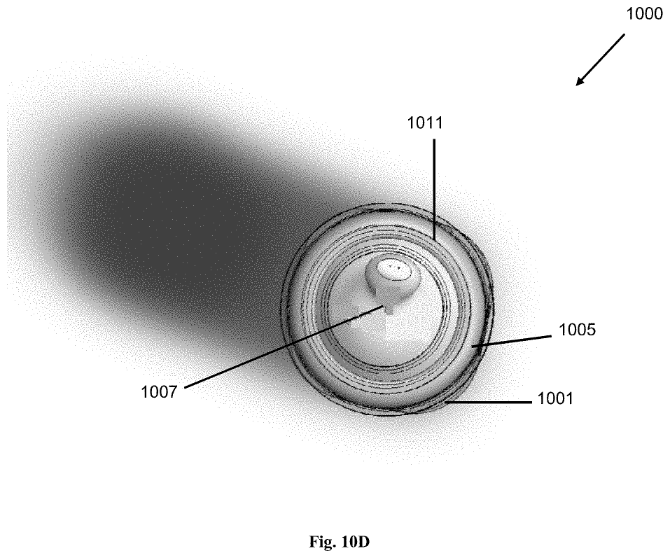

FIGS. 10A-10D illustrate various views of an assembly 1000 including a bottle 1001, nipple 1007, nipple cover 1009 and a nipple adapter 1005 in accordance with at least one embodiment of the present disclosure. Also illustrated is a protruding element 1012 of the nipple cover 1009 and a recess 1013 in a bottom surface of a bottle 1001, the both of which may be used to stack the bottles in accordance with the disclosure herein.

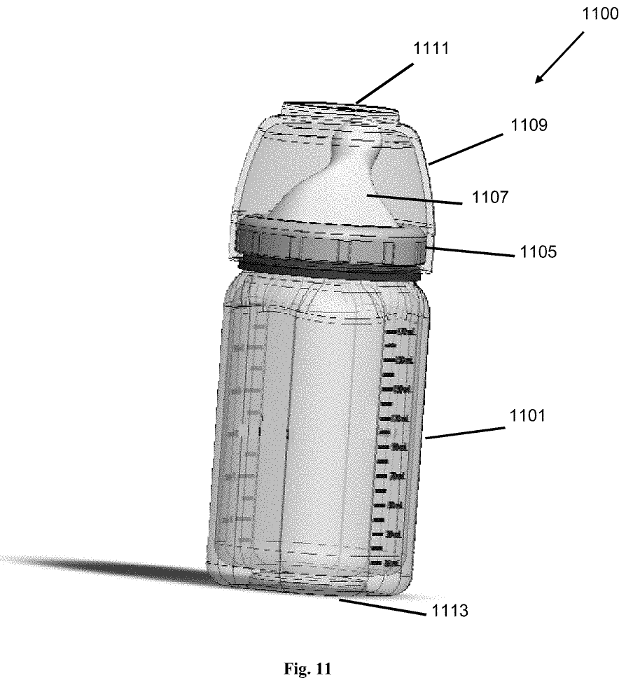

FIG. 11 illustrates a perspective view of an assembly 1100 including a bottle 1101, nipple 1107, nipple cover 1109 and a nipple adapter 1105 in accordance with at least one embodiment of the present disclosure. Also illustrated is a protruding element 1111 of the nipple cover 1109 and a recess 1113 in a bottom surface of a bottle 1101, the both of which may be used to stack the bottles in accordance with the disclosure herein.

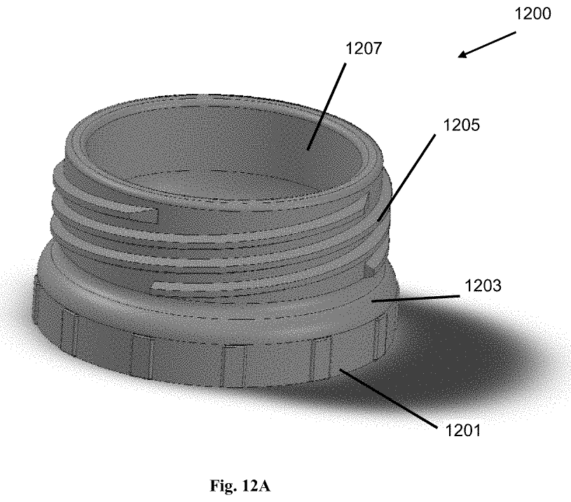

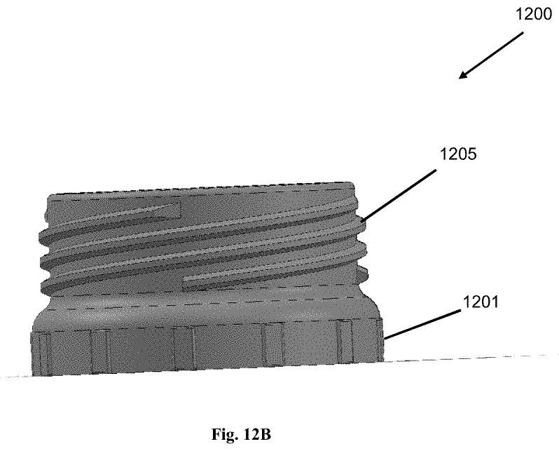

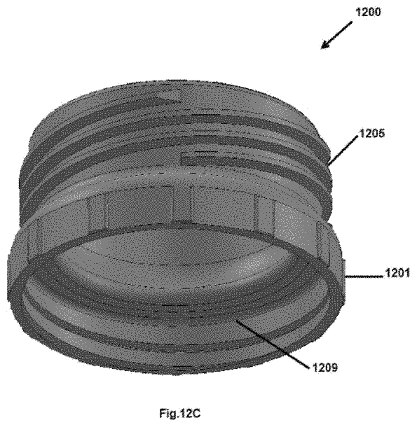

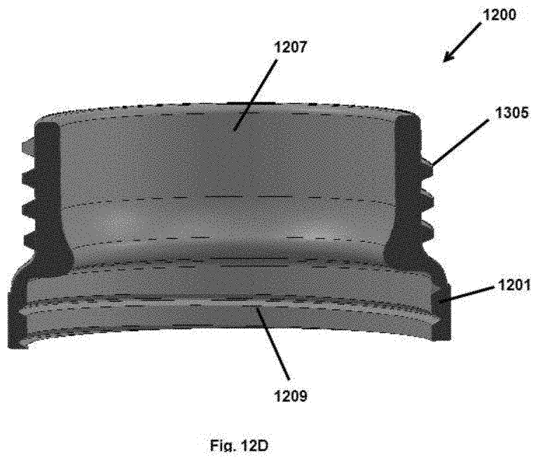

FIGS. 12A-12D illustrate various views of a breast pump adapter 1200 for a bottle in accordance with at least one embodiment of the present disclosure. In particular, FIGS. 12A-12C provide various perspective views of the breast pump adapter 1200 and FIG. 12D provides a cross-sectional view of the breast pump adapter 1200. A breast pump adapter 1200 may be configured to connect a bottle such as bottle 100, 200 with a breast pump. Example breast pumps may include those manufactured by Medela®, Spectra Baby®, and Phillips Avent®.

The breast pump adapter 1200 may have any suitable configuration for attaching to both a bottle and a breast pump. For example, the breast pump adapter 1200 may include a lower exterior portion 1201, an upper exterior portion 1205, an exterior flat surface 1203, an inner upper portion 1207, and a lower interior portion 1209. In some embodiments, the lower interior portion 1209 may include attachment means for engaging with the inner adapter of the bottle. For example, if the inner adapter 300 is configured with an exterior surface 303 having a male thread, the lower interior portion 1207 of the breast pump adapter 1200 may include a female thread capable of engaging with the male thread. Further, the upper exterior portion 1205 may be capable of engaging with a breast pump using a screwed attachment.

The breast pump adapter 1200 may be configured such that a woman is able to express breast milk directly into a bottle 100, 200 rather than having to pump into a temporary bottle (that is not ideally designed to be frozen), and then transfer the milk from the temporary bottle into a plastic bag for storage.

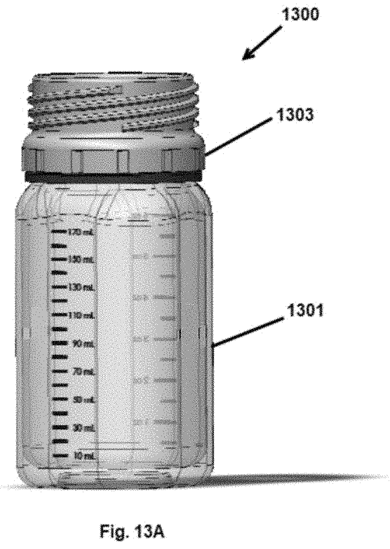

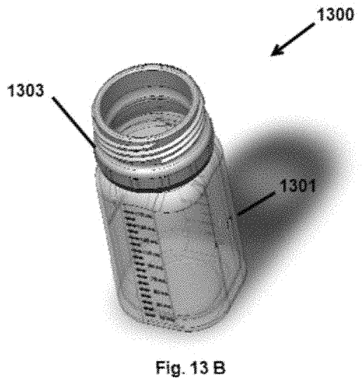

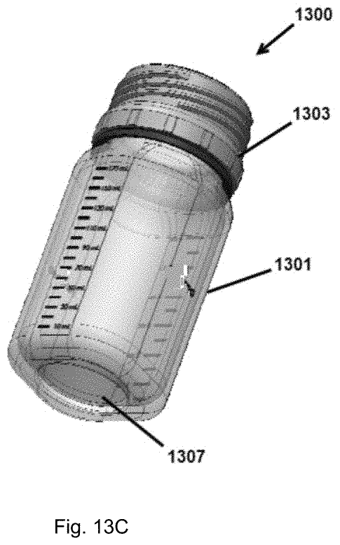

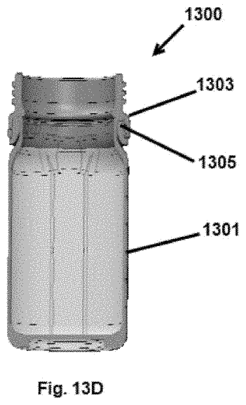

FIGS. 13A-13D illustrate various views of an assembly 1300 including a bottle 1301 and breast pump adapter 1303 in accordance with at least one embodiment of the present disclosure. In particular, FIGS. 13A-13C illustrate various perspective views of the assembly 1300, and FIG. 13D illustrates a cross-sectional view of the assembly 1300. As illustrated, the bottle 1301, may include a bottom recess 1307. Further, as illustrated in FIG. 13D, the breast pump adapter 1303 may be configured to screw 1305 onto an inner adapter of the bottle 1301.

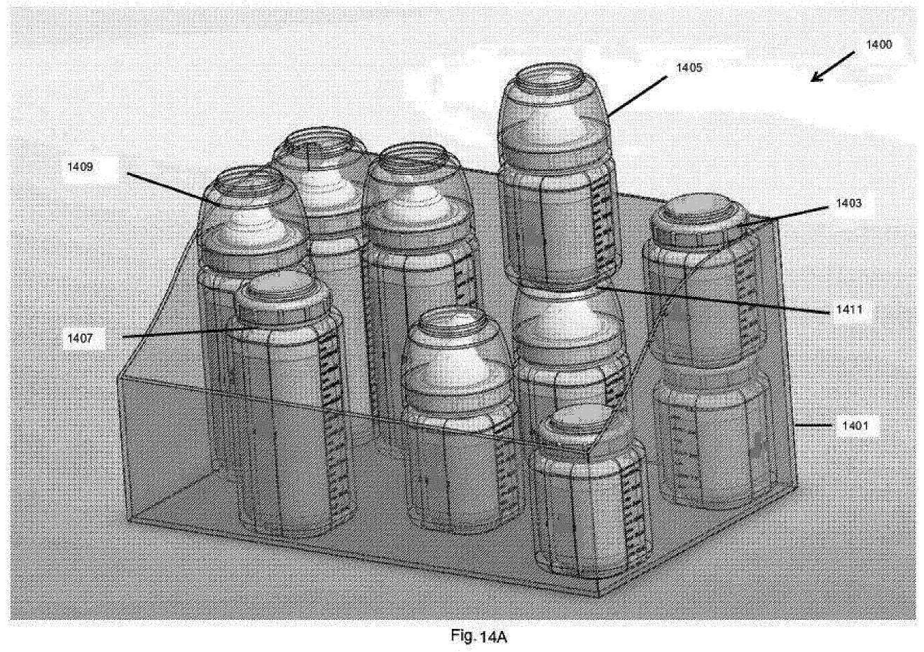

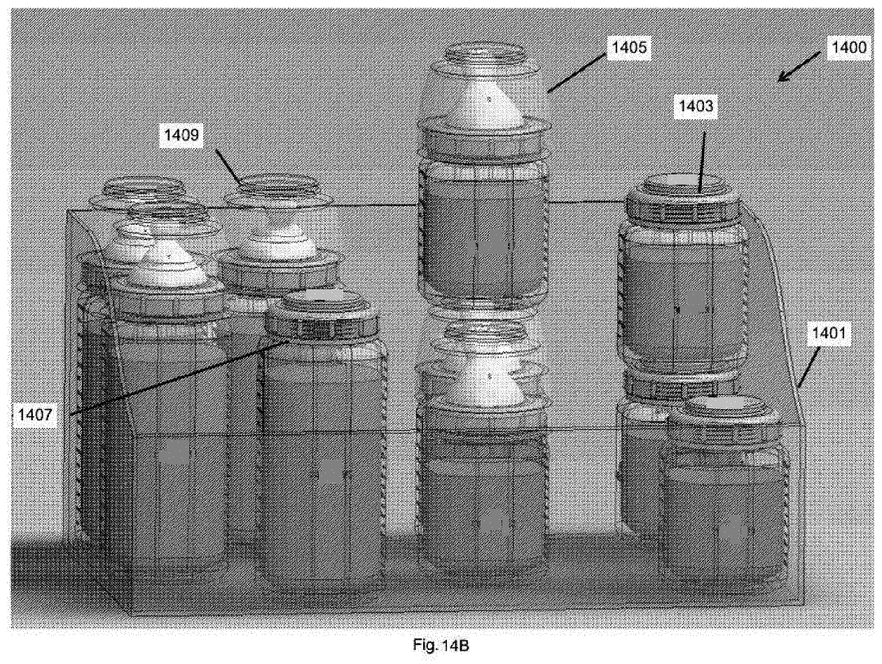

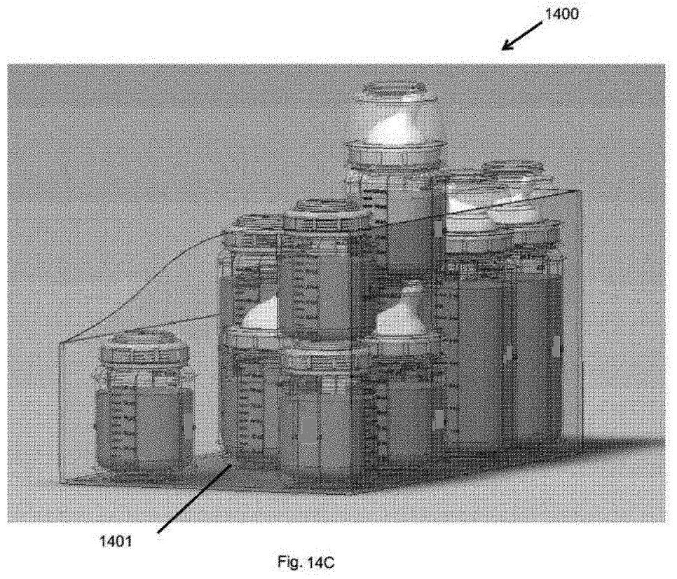

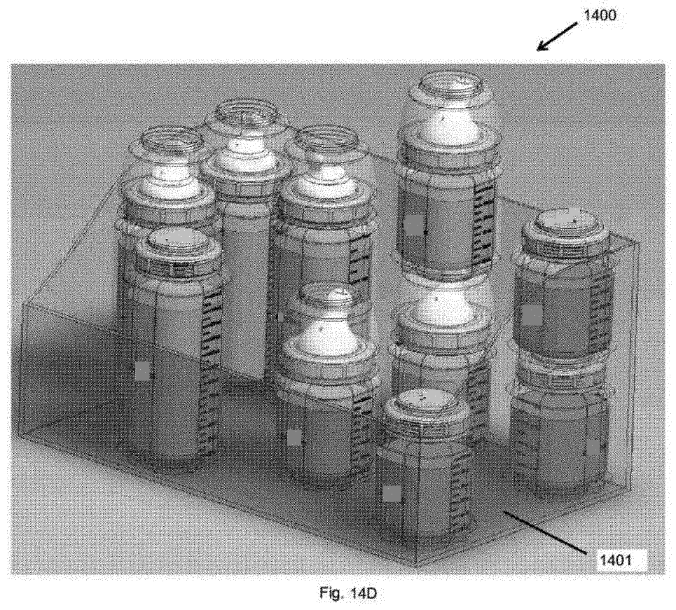

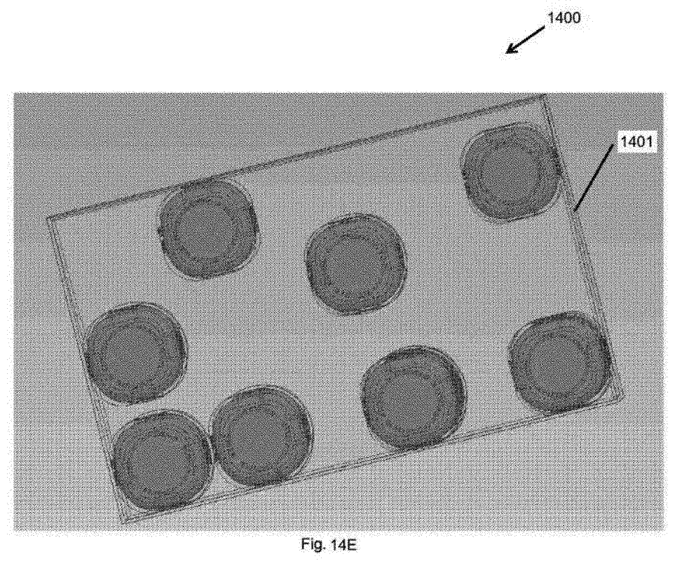

FIGS. 14A-14E illustrate various views of an assembly 1400 including various bottles, and a storage element 1401 in accordance with at least one embodiment of the present disclosure. The storage element 1401 may be configured for use within a freezer, or fridge. In some embodiments, the storage element 1401 may be formed of plastic. In some embodiments, the storage element 1401 is formed of hard, thermo-plastic.

In some embodiments, the storage element 1401 may be configured to hold at least 120 ounces of milk. The storage element 1401 may have a rectangular shape with a first side having a height greater than a second side spaced apart from the first side. Alternatively, the storage element 1401 may have a shape with rounded edges. The first side may be configured to be positioned along the rear of the freezer. Having a first side with a greater height than a second side may allow for the easy arrangement of bottles within the storage element 1401. Further, the described configuration may help assist in simplifying the first in, first out inventory management process that parents otherwise must keep track of and organize on their own.

Various bottles may be arranged within the storage element 1401 including bottles ready for feeding, having a nipple and nipple cover 1409, 1405, and bottles with a flat cap 1407, 1403. In some embodiments, bottles may be of an 8-ounce capacity 1407, 1409, or of a 4-ounce capacity 1403, 1405. In some embodiments, bottles may be of a 6-ounce capacity. In some embodiments, bottles may be stacked by a protruding element of a nipple cover or flat cap of a first bottle engaging 1411 with a recessed element positioned along a bottom surface of a second bottle.

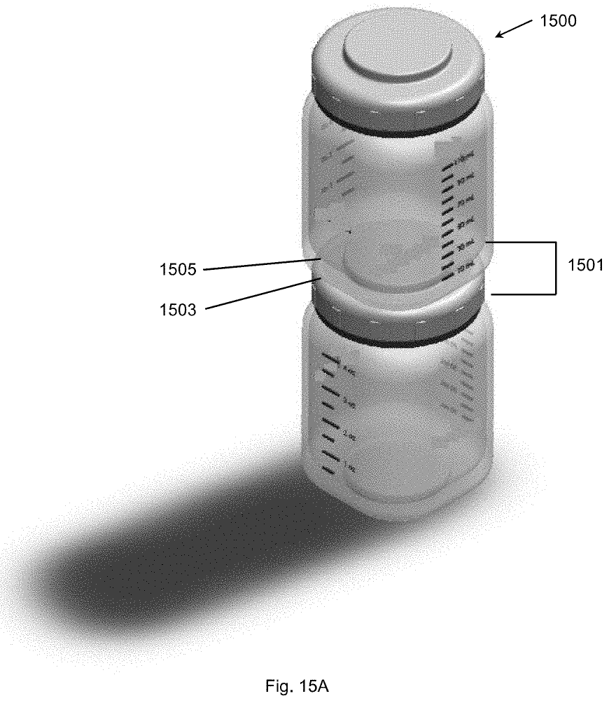

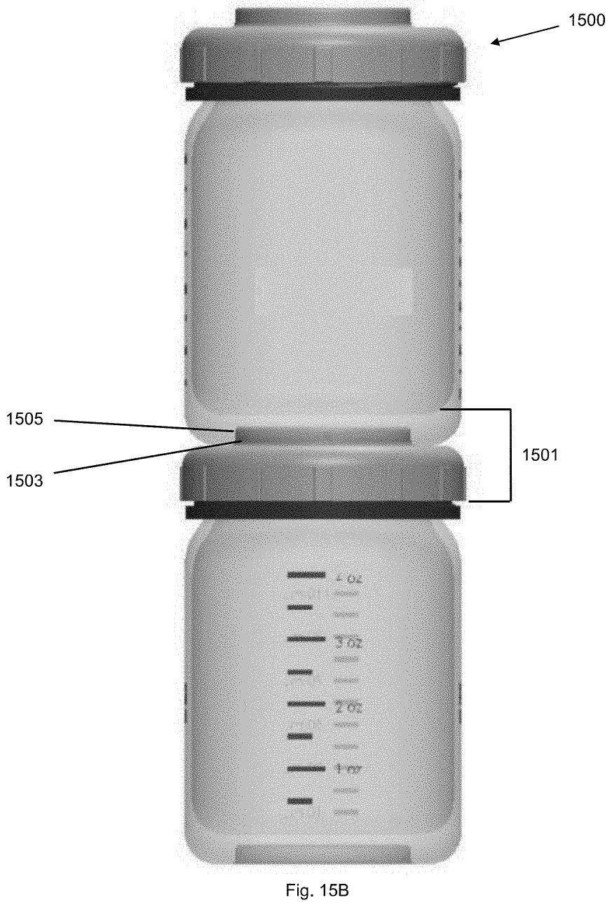

FIGS. 15A-15B illustrate various views of an assembly 1500 including stacked bottles in accordance with at least one embodiment of the present disclosure. As illustrated, in a stacking region 1501, a protruding element 1505 from the flat cap engages with a recessed portion 1503 of the bottom of a bottle.

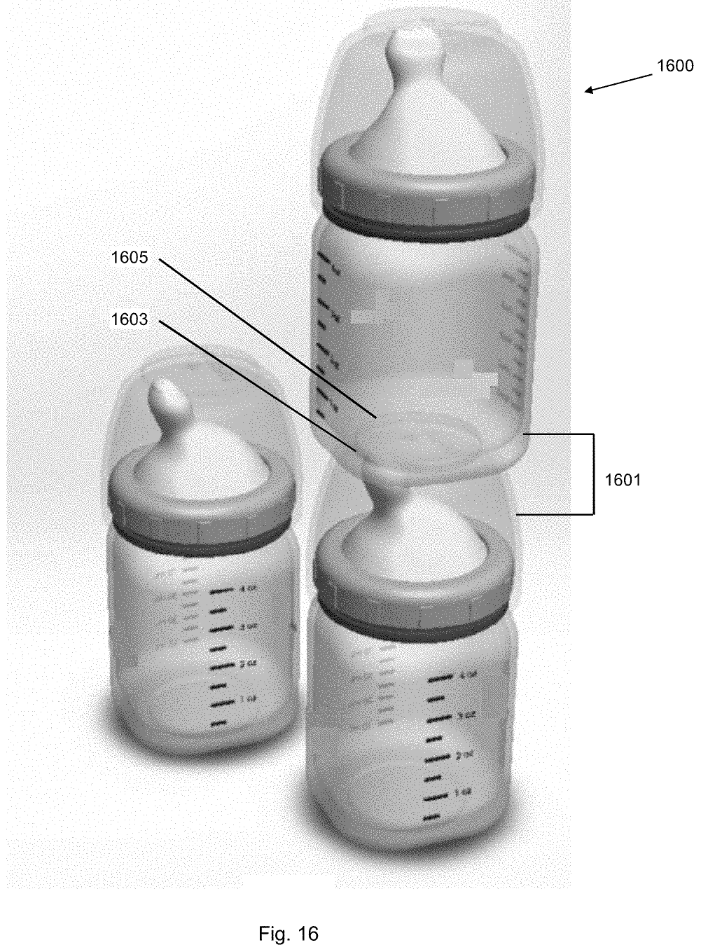

Similarly, FIG. 16 illustrates various views of an assembly 1600 including stacked bottles in accordance with at least one embodiment of the present disclosure. As illustrated, in a stacking region 1601, a protruding element 1605 from the nipple cover engages with a recessed portion 1603 of the bottom of a bottle.

The ability to stack bottles as described in the provided embodiments, and particularly FIGS. 14A-16, maximizes the amount of breast milk that can be stored in a tight freezer space and provides many advantages over the conventional systems where breast milk is commonly frozen in plastic bags in odd shapes that contribute to them sliding about in the freezer.

In some embodiments, the bottle containers will be adaptable for use in the entire pump, store, feed process. In some embodiments, the bottles may be used to store baby food, and the like.

While the invention has been disclosed in connection with preferred embodiments shown and described in detail, their modifications and improvements thereon will become readily apparent to those skilled in the art.

We claim:

1. A bottle system comprising:
a bottle body formed from molded silicone rubber comprising a bottom end having a recess, and a top end having an opening configured to receive and dispel contents, wherein the top end is spaced apart from the bottom end; and
an inner adapter composed of thermo-plastic configured to engage with the top end of the bottle body, and having a top surface configured to engage with a compressible, protruding ridge of the bottle body so as to create a tight seal.

2. The bottle system of claim 1, comprising:
a flat cap configured to engage with the inner adapter and form a closed environment, the flat cap comprising a protruding surface configured to engage with the recess of the bottle body, and wherein a bottom surface of the flat cap engages with the compressible, protruding ridge of the bottle body, wherein the protruding ridge rests on the top surface of the inner adapter.

3. The bottle system of claim 1, comprising:
a nipple configured to engage with the ridge of the bottle body.

4. The bottle system of claim 3, comprising:
a nipple adapter configured to engage with a nipple and secure the nipple to the inner adapter, wherein a bottom surface of the nipple adapter engages with the compressible, protruding ridge of the bottle body.

5. The bottle system of claim 4, comprising:
a nipple cover configured to engage with the nipple adapter and prevent the nipple from expelling liquid contained within the bottle body, the nipple cover comprising a protruding surface configured to engage with the recess of the bottle body.

6. The bottle system of claim 1, comprising:
a storage element having a first end spaced apart from a second end, the first end having a greater height than the second end, wherein the storage element is configured to store a plurality of bottles.

7. The bottle system of claim 3, wherein the nipple comprises:
an upper nipple portion having a teat; and
a lower nipple portion configured to be off centered from the upper nipple portion.

8. The bottle system of claim 3, wherein the nipple comprises:
an upper nipple portion without a vent.

9. The bottle system of claim 7, wherein the lower nipple portion further comprises a vent.

10. The bottle system of claim 1, comprising:
at least one breast pump adapter, wherein each breast pump adapter is configured to connect to a particular breast pump, each breast pump adapter having a first end designed to attach to a pump valve of the breast pump and a second end configured to attach to the inner adapter.

11. The bottle system of claim 1, wherein the bottle body comprises a volume of one of 4 ounces, 6 ounces, or 8 ounces.

12. The bottle system of claim 3, wherein the nipple comprises silicone.

13. The bottle system of claim 2, wherein the flat cap comprises thermo-plastic.

14. The bottle system of claim 4, wherein the nipple adapter comprises thermo-plastic.

15. The bottle system of claim 5, wherein the nipple cover comprises translucent thermo-plastic.

16. The bottle system of claim 1, wherein the bottle body compresses in volume responsive to liquid being extracted from the bottle body.

17. A bottle system comprising:
a plurality of bottles, each bottle among the plurality of bottles having a bottle body and an inner adapter, wherein the bottle body is formed from molded silicone rubber comprising a bottom end having a recess, and a top end having an opening configured to receive and dispel contents, wherein the top end is spaced apart from the bottom end, and the inner adapter is composed of thermo-plastic configured to engage with the top end of the bottle body, and having a top surface configured to engage with a compressible, protruding ridge of the bottle body so as to create a tight seal,
wherein each of the plurality of bottles is capable of stacking with another bottle among the plurality of bottles by way of the recess of the bottom end of the bottle body engaging with a top protruding element of one of a nipple cover or a flat cap engaged with the inner adapter of another bottle body among the plurality of bottles.

18. The bottle system of claim 17, wherein the flat cap engages with the inner adapter of the other bottle body to form a closed environment, the flat cap comprising a protruding surface configured to engage with the recess of the other bottle body, and wherein a bottom surface of the flat cap engages with the compressible, protruding ridge of the other bottle body, wherein the protruding ridge rests on the top surface of the inner adapter.

19. The bottle system of claim 17, wherein the nipple cover engages with the nipple adapter of the other bottle body, and the other bottle further comprises:
a nipple configured to engage with the ridge of the bottle body,
a nipple adapter configured to engage with a nipple and secure the nipple to the inner adapter, wherein a bottom surface of the nipple adapter engages with the compressible, protruding ridge of the bottle, and
the nipple cover is further configured to engage with the nipple adapter and prevent the nipple from expelling liquid contained within the bottle.

20. The bottle system of claim 17, comprising:
a storage element having a first end spaced apart from a second end, the first end having a greater height than the second end, wherein the storage element is configured to store the plurality of bottles.
